(19) United States
(12) Patent Application Publication
(10) Pub. No.: US 2017/0189416 A1
(43) Pub. Date: Jul. 6, 2017

(54) PHARMACEUTICAL COMPOSITION FOR TREATING BREAST CANCER

(71) Applicant: MA SHEN KAI RUEi CO., LTD., Tainan City (TW)

(72) Inventor: PO-LIANG CHEN, Taipei City (TW)

(21) Appl. No.: 14/983,756

(22) Filed: Dec. 30, 2015

(51) Int. Cl. A61K 31/575; A61K 31/122

(52) U.S. Cl. CPC A61K 31/575; A61K 31/122

(57) ABSTRACT

The present invention provides a pharmaceutical composition for treating breast cancer in a subject in need thereof, which comprises a first agent comprising at least a dehydroeburicoic acid (DeEA); and a second agent comprising at least an Antroquinonol B (AnQB); as well as each of them can be obtained from the extractions of a fruiting body or a mycelium of Antrodia camphorata wherein the first agent and the second agent shows synergistic effect for use in the treatment of breast cancer or to prevent or to reduce the risk of a breast cancer metastasizing, compared to administration of the first agent or the second agent alone.

### BACKGROUND OF THE INVENTION

1. Fields of the Invention

The present invention relates to a pharmaceutical composition for treating breast cancer in a subject in need thereof, comprising a first agent and a second agent obtained individually from extractions of Antrodia camphorate; especially relates a pharmaceutical composition for treating breast cancer, which comprises a first agent comprising at least a dehydroeburicoic acid (DeEA) and a second agent comprising at least an Antroquinonol B (AnQB).

2. Descriptions of Related Art

The medical term “tumor” refers to the formation of a mass by abnormal cell proliferation which even violates surrounding or distant tissue, affecting the tissue's normal physiological function. The tumors are generally determined to be benign or malignant by histopathological examination. When the healthy cell is taken over, it too can replicate more abnormal cells. The malignant tumors are called cancer. It is known that many types of cancer are caused by genetic aberrations, i.e., mutations. In recent decades, cancer has been one of the top ten causes of death of the people in Taiwan.

Cancer starts when cells begin to grow out of control. Cells in nearly any part of the body can become cancer, and can spread to other areas of the body. A malignant tumor is a group of cancer cells that can grow into (invade) surrounding tissues or spread (metastasize) to distant areas of the body.

The cancer cells grow rapidly and invade surrounding tissue, the walls of nearby blood or lymphatic vessels through bloodstream or lymph system to spread to other parts of the body. The cancer cells stop moving as they are lodged in capillaries at a distant location and divide and migrate into the surrounding tissue, then cancer cells form small tumors at the new location (called micrometastases.)

The lymph system comprises lymph which contains tissue fluid and waste products, as well as immune system cells, lymph nodes which are small, bean-shaped collections of immune system cells being deemed as important in fighting infections, and lymphatic vessels which appear like small veins and are connected with lymph nodes, except that they carry a clear fluid called lymph (instead of blood). Thus, the lymph system is one important way of distribution of cancers to be spread to other parts of the body.

Breast cancer is a malignant tumor that starts in the cells of the breast. The disease occurs almost entirely in women, but men can get it, too.

Breast cancer cells can enter lymphatic vessels in the breast and begin to grow in lymph nodes under the arm (axillary nodes), or inside the chest (internal mammary nodes) and either above or below the collarbone (supraclavicular or infraclavicular nodes).

Breast cancer is the most common cause of death in women between the ages of 45 and 55. Worldwide breast cancer constitutes 23% of all cancers with the majority of breast cancers occurring in women. Still, breast cancer is the most common cause of cancer in women in Taiwan.

Treatments for cancer can be divided into three categories, namely, surgery, chemotherapy, and radiation therapy. The treatment of surgical resection focuses on removal of the tissue where cancer occurs. However, surgical resection, chemotherapy, and radiation therapy are irreversible methods that will cause destruction of human's cells, tissues, and even organs. Thus, there is an urgent need to provide a method that can effective treat or prevent cancer and will not cause irreversible secondary damage to cancer patients.

The female breast is made up mainly of lobules (milk-producing glands), ducts (tiny tubes that carry the milk from the lobules to the nipple), and stroma (fatty tissue and connective tissue surrounding the ducts and lobules, blood vessels, and lymphatic vessels). Most breast cancers begin in the cells that line the ducts (ductal cancers). Some begin in the cells that line the lobules (lobular cancers), while a small number start in other tissues.

While the majority of new breast cancers are diagnosed as a result of an abnormality seen on a mammogram, a lump or change in consistency of the breast tissue can also be a warning sign of the disease. Heightened awareness of breast cancer risk in the past decades has led to an increase in the number of women undergoing mammography for screening, leading to detection of cancers in earlier stages and a resultant improvement in survival rates.

Breast cancer occurs due to mutations in the genes responsible for regulating the growth of breast cells which causes the cells to grow in an unregulated manner.

Usually breast cancer either begins in the cells of the milk producing glands, known as the lobules, or in the ducts. Less commonly breast cancer can begin in the stromal tissues. These include the fatty and fibrous connective tissues of the breast. In some embodiments, the breast cancer is invasive ductal carcinoma, ductal carcinoma in situ, invasive lobular carcinoma, or lobular carcinoma in situ.

A particularly aggressive form of breast cancer is characterized by amplification of the Her2 gene which results in overexpression of its protein product. Her2 stands for “human epidermal growth factor receptor 2” and is also known as ErbB-2. Approximately 15 to 20% of breast cancers have amplification of the Her2 gene.

Over time the breast cancer cells can invade nearby tissues such the underarm lymph nodes or the lungs in a process known as metastasis. The stage of the breast cancer, the size of the tumor and its rate of growth are all factors which determine the type of treatment that is offered. Because breast cancer is such a major problem of occurrence of relatively aggressive development and existing treatments have limited long-term success, it generally occurs relatively slowly over years and even decades in some cases.

Treatment options include surgery to remove the tumor, drug treatment which includes chemotherapy. The prognosis and survival rate varies widely; the five year relative survival rates vary from 98% to 23% depending on the type of breast cancer that occurs. and hormonal therapy, radiation therapy and immunotherapy.

Although the use of a few therapy has clearly improved the outcome of patients with breast cancer, an effective pharmaceutical composition for treating breast cancer in a subject in need thereof is still a clinical challenge.

Medicinal plants have been used for centuries to treat a variety of diseases and maintain health before the advent of modern medicine. The accumulation and developing knowledge of the medicinal properties of plants by personal experimentation, local custom, anecdote, and folk tradition leads to the formation of numerous traditional medical systems and therapies, including traditional Chinese medicine (TCM).

Antrodia camphorata and the mycelia products therefrom or thereof possesses edible high value with various excellent functions not only in medicinal such as having anti-oxidant, antihypersensitive and immunostimulatory effects but also the capability of improving physical health by its own anticancer activity, reduced treatment-related symptoms and other side effects similar to medical efficiency of the wild fruiting bodies.

Consequently, many products made from Antrodia camphorata and/or comprising especially the active ingredient extracted form thereof such as Antrodia oil, Antrodia extraction, Antrodia combination and so on, are broadly used in various medicine, health care applications and also Antrodia camphorata and wild cattle camphor thus has been listed as one of the biological treasure in recent years by the Taiwan Government.

Antrodia camphorata is a non-mesh skirt bacteria, an endemic fungus, and grows in the internal heartwood (or the dark/humid wood surface) of Cattle camphorin the mountainous region of Taiwan, altitude 450-2000 meters mountain forest. It is also perennial mushroom fungus and only grows in the inner wall of a wood trunk decayed decades or more, or the lodging of the dead wood wet surface of a Cattle camphor, particularly Cinnamomum kanehirai.

Antrodia camphorata is rich in Triterpenoids, immunostimulatory polysaccharides such as -D-glucan polysaccharides, Adenosine, Nicotinic acid, SOD (superoxide dismutase enzymes), Steroids, Vitamin, essential minerals and other pharmaceutically active principles.

In addition, Antrodia extraction and/or Antrodia oil also contains much important nutrients to the human body, for examples, oleic acid, palmitic acid, linoleic acid, palmitoleic acid, linolenic acid, stearic acid, meat, beans Qu acid, arachidic acid, behenic acid, tetracosanoic acid, n-heptadecyl acid, n-heptadecenoic acid, vitamin A, vitamin B, vitamin E and minerals; as well as it also can inhibit tumor metastasis, reduce the incidence of coronary heart disease, improve immunity and other effects.

Thereby, Antrodia camphorata shows various excellent functions such as detoxification, hypoglycemic effects, reducing blood pressure, improving anti-cancer effect, inhibition of histamine release effect, enhancing anti-inflammatory effect; enhancing immunity, increasing cell viability, eliminating free radicals, promoting liver cell regeneration, lowering down alanine aminotransferase, and in addition to even enhancing the phagocytic capacity of macrophages as well as having the capability in improving physical health.

Although the extracts of Antrodia camphorates, and anticancer agents or compositions comprising thereof, have medical effects as described above and have been attracted attention of people widespread, it still cannot be used as a normal anti-tumor agent for breast cancer or used as sole-therapy drug for the treatment of breast cancer, due to it is still not clear exactly know what is the particular active ingredient or bioactive composition presented therein.

As to medicine and pharmacy science, there is need of a drug for treatment or amelioration of breast cancer with satisfactory effect, there is urgent need to develop a pharmaceutical composition which is capable of solving the defects of anti-breast cancer drugs existed in the prior arts and providing excellent medical effects of treating breast cancer.

### SUMMARY OF THE INVENTION

In view of defects and problems as above-mentioned, the inventors of the present application conduct several researches with respect to those problems remained in conventional technologies of prior arts.

As results, while an pharmaceutical composition or combination comprising active ingredients or components such as the dehydroeburicoic acid (DeEA) and/or Antroquinonol B (AnQB) obtained from the extraction Antrodia camphorates, is used in the treatment and/or prevention of breast cancers, it is surprising to find that unexpected excellent effects are achieved as compared to the effects offered by the prior arts of traditional anti-cancer agent.

The unexpected excellent effect include for examples, at least reducing or regulating carcinogenic activity, preventing proliferation or even reversing of cancer cells, and also treating and/or preventing cancer and tumor metastasis.

In addition to those unexpected excellent effects, it also found not only having excellent characteristics chemistry, biology, mechanical science and physical science, but also having good performance of transmittance and transportation.

Further, it is also found that the pharmaceutical composition or combination is very easy for the user uptaking, in short digestion and absorption, and can be used as drugs or adjuvant for the treatment and/or prevention of cancers, especially can inhibit the prevention and treatment of cancer with efficacy while applied to specific cancer cells.

Thus, the present invention is achieved.

Namely, according to an aspect of the present invention, provided is a pharmaceutical composition for treating breast cancer in a subject in need thereof, which comprises (1) a first agent comprising at least a dehydroeburicoic acid (DeEA) obtained from Extractions of a fruiting body or a mycelium of Antrodia camphorata; (2) a second agent comprising at least an Antroquinonol B (AnQB) obtained from the Extractions of a fruiting body or a mycelium of Antrodia camphorata; wherein the first agent and the second agent shows synergistic effect for use in the treatment of breast cancer or to treat, prevent or to reduce the risk of a breast cancer metastasizing, compared to administration of the first agent or the second agent alone.

According to another aspect of the present invention, further provided is a pharmaceutical composition as described above, wherein the first agent is a dehydroeburicoic acid (DeEA) in a pharmaceutically effective amount for use in the treatment of breast cancer or to treat, prevent or to reduce the risk of a breast cancer metastasizing; and/or the second agent is an Antroquinonol B (AnQB) in a pharmaceutically effective amount for use in the treatment of breast cancer or to treat, prevent or to reduce the risk of a breast cancer metastasizing.

Further, according to one aspect of the present invention is to provide a pharmaceutical composition as described above, wherein the first agent and the second agent are in the form of a botanical drug substance (BDS); and may be administered to a human cancer patient in any manner selected from co-administration, a daily regimen, an episodic regimen, intravenous administration, or oral administration.

Furthermore, according to another aspect of the present invention is to provide a pharmaceutical composition as described in Claim 1, wherein the dehydroeburicoic acid (DeEA) and/or Antroquinonol B (AnQB) is present in an approximate amount of between 10 mg and 350 mg, in the ratio (DeEA:AnQB) within the ranges of about 3:1 to about 1:3, about 9:1 to about 1:9, or about 23:16 to about 16:23.

Additionally, according to one aspect of the present invention is to provide a pharmaceutical composition as described above, which further comprises a pharmaceutically acceptable ingredient comprising a vehicle, a carrier, a diluent or an excipient; wherein the excipient comprises an ingredient selected from the group consisting of lactose, sucrose, a mannitol, sorbitol, maize starch, wheat starch, rice starch, potato starch, gelatin and tragacanth.

Besides, according to another one aspect of the present invention is to provide pharmaceutical composition as described above, wherein further comprises at least one additive selected from the group consisting of absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, bactericides, sweeteners, solubilizers, wetting agents, and a mixture thereof.

### DETAILED DESCRIPTION OF THE PREFERRED EMBODIMENT

In order to make the spirit and content of the present invention more completely and easily to be understood, various examples of the present invention, particularly various specific embodiments are described in more detailed hereinafter.

However, a skilled person having general knowledge in this technical field pertains to the present invention, shall understand that the present invention is of course not limited to these examples only, and it is possible to achieve the invention by means of taking advantage of other features of function, efficiency or processes which are the same or equal with the present invention.

First, the descriptive instructions or definitions with respect to a term or a description word or phase particularly used in this specification are separately described below.

Unless defined otherwise, all technical and scientific terms used herein have the same meaning as is commonly understood by one of skill in the art to which the claimed subject matter belongs.

It is to be understood that the foregoing general description and the following detailed description are exemplary and explanatory only and are not restrictive of any subject matter claimed. In this application, the use of the singular includes the plural unless specifically stated otherwise.

It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

The section headings used herein are for organizational purposes only and are not to be construed as limiting the subject matter described.

As used herein, a term of “treatment (or treating)” refers to implementing a preventive, curative or palliative disposal for achievement of pharmaceutical and/or physiological effects to an individual subject or a patient having a certain medical condition, symptoms, disease, disorder or an initial condition, in order to partially or completely reduce the severity, delay the occurrence process, and/or inhibit one or more symptoms of the medical condition, abnormal and/or the probability of occurrence of behavior disorders.

As used herein, a term of “effective amount” refers to while a medical drug for cancer is administered (administering, or administration) directly or indirectly in a certain amount, and an effect of reducing the number of cancer cells, or particular purpose of treating or preventing a cancer is shown.

The said “certain amount” is so called effective amount.

As used herein, “individual (subject)” or “patient (patient)” can be used interchangeably with one another. The “individual (or individual subject)” or “patient” means, including but not limited to, a human that can accept a compound and/or method for treatment.

Except as otherwise specifically stated that the “individual (or individual subject)” or “patient” may comprise males and females of both sexes. Also, a preferable individual or patient for treatment by using a pharmaceutical composition and/or a method of the present invention is preferably a human.

In this context, the value of the parameter used for defining the scope of the present invention, in essence, inevitably contain standard deviation caused due to individual test methods, and thus it is mostly expressed by an approximate value of number.

However, in particular the implementation of Examples, the value is presented as precisely as possible. In this document, “approximate (or about)” is determined by the skilled person having the usual knowledge of the present invention generally pertains to.

Generally, as used herein, “about” includes an amount that would be expected to be within experimental error. Hence “about 10 μg” means “about 10 μg” and also “10 μg”. It also refers to the actual value which falls in the range of an acceptable standard deviation including the exact amount, for example, the actual value is expressed by a ±10%, it means within a range, ±5%, ±1%, or ±0.5% of a particular value.

According to the present invention, a pharmaceutical composition for treating breast cancer in a subject in need thereof is provided. The pharmaceutical composition comprises a first agent and a second agent obtained individually from extractions of Antrodia camphorate.

According to the present invention, the first agent used in the pharmaceutical composition for treating breast cancer, may comprise, but are not limited to at least a dehydroeburicoic acid (DeEA). In this context, the “dehydroeburicoic acid (DeHBA)” generally has a chemical structure usually represented by Formula (2) as shown below, with molecular formula of C₃1H₄₈O₃ and molecular weight of 468.7.

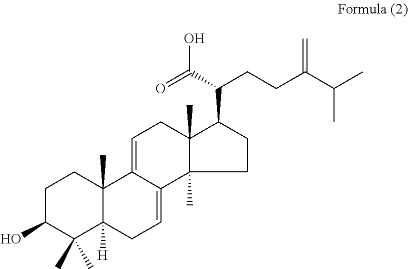

According to the present invention, dehydroeburicoic acid (DeEA) used as the first agent in the pharmaceutical composition for treating breast cancer, may be obtained by purification or isolation from the Extractions of Antrodia camphorata, which is rich in dehydroeburicoic acid (DeEA) and another bioactive ingredients for cancer treatment.

It is reported that the extractions extracted from the Antrodia camphorata comprise many effective bio-ingredients in cancer treatment, for example, Sesquiterpenoids (sesquiterpene compounds), Diterpenoids, Triterpenoids, Steroids, furan ring structure such as five member (Furan) or pyrazolyl class (Pyrrole), lignan compound (Lignoids), benzene compounds (Benzenoids), superoxide dismutase and amino acids and the like.

In the extractions extracted from the Antrodia camphorata, the Sesquiterpenoids that can be used as a cancer-treatment-effective bio-ingredient may comprise, but are not limited to, for example Antrocin and the like.

Further, in the extractions extracted from the Antrodia camphorata, the Diterpenoids (diterpene compounds) that can be used as a cancer-treatment-effective bio-ingredient may comprise, but are not limited to, for example 19-hydroxylabda-8(17)-en-16,15-olide, 3β,19-dihydroxylabda-8(17),11E-dien-16,15-olide, 13-epi-3β,19-dihydroxyl-abda-8(17),11E-dien-16,15-olide, 19-hydroxylabda-8(17),13-dien-6,15-olide,14-deoxy-11,12-didehydroandrograph-olide, 14-deoxy-andrographolide, pinusolidic acid and so on.

Furthermore, in the extractions extracted from the Antrodia camphorata, the Triterpenoids that can be used as a cancer-treatment-effective bio-ingredient may comprise, but are not limited to, for example CamphoratinB, camphoratin A, Antcin K, Antcin I (zhankuic acid B, 3α-hydroxy-4α-methylergost-8,24(28)-dien-7,11-dione-26-oic acid), camphoratin E, antcin H (zhankuicacid C, 3α,12α-dihydroxy-4α-methylergost-8,24(28)-dien-7,11-dione-26-oic acid), methyl antcinate H (3α,12α-dihydroxy-7,11-dioxo-4α-methylergost-8,24(28)-dien-26-oate), zhankuic acid E, camphoratin C, camphoratin H, camphoratin I, antcin A (1,4α-methylergost-8,4(8)-diene-3,11-dion-26-oic acid), camphoratin J, methyl antcinate A (methyl 4α-methylergost-8,24(28)-dien-3,11-dion-26-oate), antcin E (3,11-dioxo-4α-methylergost-8,14,24(28)-trien-26-oic acid), antcin C(7β-hydroxy-4α-methylergost-8,24(28)-diene-3,11-dion-26-oic acid), camphoratin G, antcin F (3,11-dioxo-7β-hydroxy-4α-methylergost-8,14,24(28)-trien-26-oic acid), camphoratin D, camphoratin F, methyl antcinate G (7α-acetoxy-3,11-dioxo-4α-methylergost-8,24(28)-dien-26-oate), antcin B (zhankuicacid A, 4α-methylergost-8,24(28)-dien-3,7,11-trion-26-oic acid), antcin D (zhankuicacid F, 14-hydroxy-4α-methyl-3,7,11-trioxoergost-8,24(28)-dien-26-oic acid), methyl antcinate B (methyl 4α-methylergost-8,24(28)-dien-3,7,11-trion-26-oate), zhankuic acid D, eburicol (24-methylenedihydrolanosterol), eburicoic acid (35),7 sulphurenic acid, versisponic acid D, dehydroeburicoic acid, dehydrosulphurenic acid, 15α-acetyldehydrosulphurenic acid, 3β,15α-dihydroxylanosta-7,9(11),24-triene-21 oicacid, epi-friedelinol and so on.

Steroids generally comprise β-Sitosterol, stigmasterol (44),16 ergosterol peroxide, ergosterol D, ergosterol, β-sitostenone, ergosta-4,7,8(14),22-tetraen-3-one, ergosta-2,4,8(14),22-tetraen-3-one an so on.

Additionally, in the extractions extracted from the Antrodia camphorata, the Furan ring structures such as five (Furan) class or pyrazolyl (Pyrrole) class that can be used as a cancer-treatment-effective bio-ingredient may comprise, but are not limited to, for example Antrocinnamomin C (3-isobutyl-4-(4-hydroxyphenyl)furan-2,5-dione), 3-isobutyl-4-[4-(3-methyl-2-butenyloxy)phenyl]furan-2,5-dione antrocinnamomin D (2-hydroxy-3-isobutyl-4-[4-(3-methylbut-enyloxy) phenyl]-2H-furan-5-one), cis-3-(4-hydroxyphenyl)-4-isobutyl-dihydrofuran-2,5-dione, dimethyl-2-(4-hydroxyphenyl)-3-isobutyl-maleate, 3-(4-hydroxyphenyl)-4-isobutyl-1H-pyrrole-2,5-dione, 3-iso-utyl-4-[4-(3-methyl-2-butenyloxy)phenyl]-1Hpyrrole-2,5-dione (antrodin B, camphorataimideB), trans-3-isobutyl-4-[4-(3-methyl-2-butenyloxy)phenyl]pyrrolidine-2,5-dione, antrocinnamomin B (3-isobutyl-4-(4-hydroxyphenyl)-1H-pyrrol-1-ole-2,5-dione), 3-isobutyl-4-[4-(3-methyl-2-butenyloxy)phenyl]-1H-pyrrol-1-ol-2,5-dione (antrodin C, camphorataimide C), antrocinnamomin A(3-isobutyl-4-[4-(3-methyl-2-butenyloxy)phenyl]-1H-pyrrol-1-acetoxyl-2,5-dione), trans-1-hydroxy-3-(4-hydroxyphenyl)-4-isobutylpyrrolidine-2,5-dione, 3R,4S-1-hydroxy-3-isobutyl-4-[4-(3-methyl-2-butenyloxy)phenyl]pyrrolidine-2,5-dione), antrodin D (camphorataimide D, 3R, 4R-1-hydroxy-3-isobutyl-4-[4-(3-methyl-2-butenyloxy)phenyl]pyrrolidine-2,5-dione), antrodi-oxolanone and so on.

Besides, in the extractions extracted from the Antrodia camphorata, the Lignoids that can be used as a cancer-treatment-effective bio-ingredient may comprise, but are not limited to, for example (+)-sesamin, (−)-sesamin, 4-hydroxysesamin, Aptosimon and so on.

In the extractions extracted from the Antrodia camphorata, the Benzenoids that can be used as a cancer-treatment-effective bio-ingredient may comprise, but are not limited to, for example 1,4-dimethoxy-2,3-methylene-dioxy-5-methylbenzene, methyl 2,5-di-ethoxy-3,4-methylene-dioxybenzoate, 4,5-dimethoxy-2,3-methylene-dioxybenzoic acid, 2,4,5-trimethoxybenzaldehyde, 2,3-methylene-dioxy-6-methylbenzene-1,4-diol, 2,4-dimethoxy-6-methylbenzene-1,3-diol, benzocamphorin C, 5-methylbenzo[1,3]-dioxole-4,7-dione, 2-methoxy-5-methyl[1,4]benzo-quinone, 2,3-dimethoxy-5-methyl[1,4]benzoquinone, isobutylphenol, 2,3,4,5-tetramethoxybenzoylchloride, 2,2,5,5,-tetra-methoxy-3,4,3,4,-bis(methylenedioxy)-6,6,-dimethylbiphenyl benzocamphorin E, benzocamphorin D, antrocamphin A, antrocamphin B, benzocamphorin A, benzocamphorin B and so on.

More specifically, in the extractions extracted from the Antrodia camphorata, the other compounds that can be used as a cancer-treatment-effective bio-ingredient may comprise, but are not limited to, for example α-Tocospiro B, methyloleate, antroquinonol, antroquinonol B, 4-acetylantroquinonol B, adenosine, cordycepin and so on.

In some embodiments, the first agent may comprise sub-ingredient which is other than the main component of dehydroeburicoic acid (DeEA) and for example, may be one compound selected form Sesquiterpenoids, Diterpenoids, Triterpenoids, Steroids, furan ring structure such as five member (Furan) or pyrazolyl class (Pyrrole), lignan compound (Lignoids), benzene compounds (Benzenoids), superoxide dismutase and amino acids and the like. Preferably, the sub-ingredient of the first agent may comprise, but are not limited to at least one selected from the group consisting of antroquinonol, antrocinnamonin A, antrocinnamonin B, antroquinonol D, zhankuic acid A, zhankuic acid C, antcin K, antcin C, and a mixture thereof.

According to the present invention, the second agent used in the pharmaceutical composition for treating breast cancer, may comprise, but are not limited to at least Antroquinonol B (AnQB). In this context, the “Antroquinonol B (AnQB)” generally has a chemical structure usually represented by Formula (1) as shown below, with molecular formula of C₂₄H₃₈O₅ and molecular weight of 406.

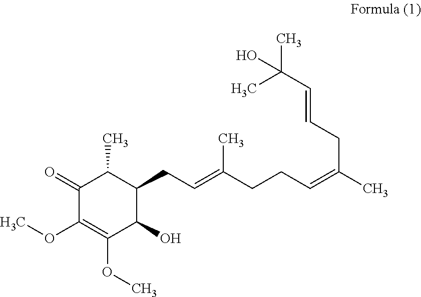 [SMILES: COC1=C(OC)[C@H](O)[C@H](C/C=C(\C)CC/C=C(/C)C/C=C/C(C)(C)O)[C@@H](C)C1=O]

In some embodiments, the second agent may comprise Antroquinonol B (AnQB) as a main ingredient and a sub-ingredient such one compound selected form Sesquiterpenoids, Diterpenoids, Triterpenoids, Steroids, furan ring structure such as five member (Furan) or pyrazolyl class (Pyrrole), lignan compound (Lignoids), benzene compounds (Benzenoids), superoxide dismutase and amino acids and the like. Preferably, the sub-ingredient may comprise, but are not limited to at least one selected from the group consisting of antroquinonol, antrocinnamonin A, antroquinonol D, dehydrosulphurenic acid, zhankuic acid A, zhankuic acid C, antcin K, antcin C, and a mixture thereof.

According to the present invention, the extractions used for obtaining the first agent and/or the second agent of the pharmaceutical composition of the present invention, is not particularly limited, for example, can be isolated from the fruiting body or mycelium of Antrodia camphorata by using a conventional purification method well known in prior arts.

Whether raw materials are the fruiting body or mycelium of Antrodia camphorata or not, an extraction method suitable for use in general, includes non-polar solvent extraction, highly polar solvent extraction, low polar solvent extraction, high temperature extraction, lower temperature extraction method, the combination of their supercritical extraction method or the like.

For example, a solvent suitable used for extracting Antrodia camphorata typically includes water, inorganic solvents, organic solvents and the like. The organic solvents used in the present invention may include, but not limited to, alcohols such as methanol, ethanol or propanol, esters such as ethyl acetate, alkanes such as hexane, or halogenated alkanes such as chloromethane, chloroethane. Among them, water and ethanol are preferred, and ethanol is particularly preferred.

Further, extraction temperature suitably used for extracting Antrodia camphorata in the present invention is generally not particularly limited, for example, it can be conducted at below 0° C., further it also may be conducted in the lower temperature range of 0° C. to 40° C., or at a higher temperature range of above 50 t to 150° C.

In illustrative embodiments, the Antroquinonol B (AnQB) and dehydroeburicoic acid (DeHBA) may be obtained by isolation and/or purification processes from the extractions of Antrodia camphorata. The isolation and/or purification processes used in the present invention may include, but not limited to, liquid chromatography, gas chromatography, gas-liquid chromatography, high-performance liquid chromatography (HPLC) and the likes.

Particularly, the Extraction A of Antrodia camphorata containing the first agent and/or the second agent used in the pharmaceutical composition of the present invention, was preferably obtained by by using a specific extracting method comprising steps of (A) extracting fruiting bodies of Antrodia camphorata with hot water at a temperature in a range of 45° C.˜100° C. to obtain Extractions HW; (B) extracting the residues HW by a fractional distillation to obtain Extractions FD which are collected from a condensation liquid in a fractional distillation apparatus; (C) extracting the residues FD by immersing with a low polar solvent at least for 4 hours to obtain Extractions LPS; (D) extracting the residues LPS through a cryo-condensation process by dropping a iced ethanol/water with a temperature in a range of 0° C.˜15° C. to obtain Extractions IEW; (E) extracting the residues IEW through a SCF (supercritical fluid extraction) by using CO₂ as a solvent at a temperature of 31.26° C. and a pressure of 72 atm to obtain Extractions SCE

According to the present invention, the effects such as treating breast cancer, inhibiting the growth of breast cancer cell and others, of Antroquinonol B (AnQB) and/or dehydroeburicoic acid (DeHBA) may be tested by using any of the test methods used in the prior arts, for example, MTT assay of using 3-(4,5-dimethylthiazol-2-yl)-2, S-diphenyl tetrazolium bromide (MTT) to determine cell survival rates of breast cancer cell lines.

In some embodiments, through MTT assays, it is proved that Antroquinonol B (AnQB) and/or dehydroeburicoic acid (DeHBA) can decrease the survival rates of breast cancer cell lines (MCF-7 and MDA-MB-231) at the same time and half-maximal inhibitory concentration (IC50) values comes to be relatively low.

Thus, the pharmaceutical composition comprising a first agent of at least a dehydroeburicoic acid (DeHBA) and a second agent of at least a Antroquinonol B (AnQB) of the present invention is very useful in inhibiting growth of preferably breast cancer cell. Also, the pharmaceutical composition of the present invention can thus be further used in preparation of a medicinal composition for treating breast cancer, which has improved the therapeutic effects with respect to the prior arts.

Further, according to one aspect of the present invention, first agent and the second agent of the pharmaceutical composition as described above, may be made to be in the form of but not limited to a botanical drug substance (BDS); and may be administered to a human cancer patient in any of manners such as the one selected from co-administration, a daily regimen, an episodic regimen, intravenous administration, or oral administration.

According to one aspect of the present invention, the effective amount of dehydroeburicoic acid (DeEA) of the first agent and Antroquinonol B (AnQB) of the second agent existed in the pharmaceutical composition as described above, may be an pharmaceutical amount useful for treating of breast cancer, or preventing or reducing the risk of a breast cancer metastasizing.

according to some embodiments of the present invention, the effective amount of dehydroeburicoic acid (DeEA) of the first agent and Antroquinonol B (AnQB) of the second agent may individually be but not limited to 0.01 mg˜2000.0 mg. For example, it is suitable to be administrated within the range of 0.01 mg˜10.0 mg, preferably within the range of 0.01 mg˜8.50 mg, more preferably within the range of 0.01 mg˜6.50 mg, particularly preferably within the range of 0.01 mg˜5.00 mg.

Furthermore, according to one aspect of the present invention, the ratio of dehydroeburicoic acid (DeEA) and Antroquinonol B (AnQB) in the pharmaceutical composition as described above, shown as (DeEA:AnQB) may be but not limited to within the ranges of about 1.0:1.0 to about 1.0:20.0. For example, it is suitable to be administrated within the range of about 1.0:1.0 to about 1.0:15.0, preferably within the range of about 1.0:1.0 to about 1.0:9.0, more preferably within the range of about 3.0:1.0 to about 1:9.0, particularly preferably within the range of about 9.0:1.0 to about 1.0:9.0.

Additionally, according to another one aspect of the present invention, the pharmaceutical composition may further comprises a pharmaceutically acceptable ingredient a vehicle, a carrier, a diluent or an excipient. For example, the excipient suitable used in the present invention is an ingredient, a compound or a component selected from the group consisting of lactose, sucrose, a mannitol, sorbitol, maize starch, wheat starch, rice starch, potato starch, gelatin and tragacanth, or composition or combination thereof.

Besides, according to another one aspect of the present invention, the pharmaceutical composition may further comprise an additive. For example, the additive suitable used in the present invention is at least an ingredient, a compound or a component selected from the group consisting of absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, bactericides, sweeteners, solubilizers, wetting agents, and a mixture thereof.

In some embodiments, the pharmaceutical compositions of the present invention may be formed in a liquid formulation which is suitable for use in oral administration, for example, oral suspensions, emulsions, micro-emulsions, and/or curing liquid (elixirs). In the case of the liquid formulation, the active ingredients of the pharmaceutical compositions of the present invention may be further blended with various formulations, for example, sweetening or flavoring agents, coloring matter or dyes, if necessary, it may be further blended with emulsifying and/or suspending agents, or such as water, alcohol, propylene glycol, glycerin and other diluents, or maintain buffer pH values.

Besides, in other embodiments, the formulations comprising a liquid pharmaceutical composition of the present invention may be prepared into sterile injectable solutions or suspensions; for example, it may be manufactured into a solution that is suitable for intravenous injection, intramuscular injection, it in intraperitoneal injection, or subcutaneous administration, and others.

Diluents suitable for use in a sterile injectable solution or suspension described above, for example, may include, but are not limited to, 1,3-butanediol, mannitol, water, Ringer's solution, isotonic chloride sodium; optionally natural oils or fatty acids acceptable in pharmacy, such as oleic acid, glycerol derivatives, or such as olive oil or canola oil, and the like.

The present invention is further explained in the following embodiment illustration and examples. Those examples below should not, however, be considered to limit the scope of the invention, it is contemplated that modifications will readily occur to those skilled in the art, which modifications will be within the spirit of the invention and the scope of the appended claims.

### EXAMPLES

The details of the examples for the present invention are described as follows.

### Example 1 Extraction A Obtained from Antrodia camphorata

Firstly, an Extraction A was obtained by extracting fruiting bodies of Antrodia camphorata by using a specific method comprising steps of (A) extracting fruiting bodies of Antrodia camphorata with hot water at a temperature in a range of 45° C.˜100° C. to obtain Extractions HW; (B) extracting the residues HW by a fractional distillation to obtain Extractions FD which are collected from a condensation liquid in a fractional distillation apparatus; (C) extracting the residues FD by immersing with a low polar solvent at least for 4 hours to obtain Extractions LPS; (D) extracting the residues LPS through a cryo-condensation process by dropping a iced ethanol/water with a temperature in a range of 0° C.˜15° C. to obtain Extractions IEW; (E) extracting the residues IEW through a SCF (supercritical fluid extraction) by using CO₂ as a solvent at a temperature of 31.26° C. and a pressure of 72 atm to obtain Extractions SCF.

Subsequently, Extractions HW, Extractions FD, Extractions LPS, Extractions IEW and Extractions SCF were mixed uniformly to form a mixture denoted as Extraction A. In the Extraction A

### Example 2 Ex Vivo Survival Assay for Anti-Breast Cancer Effects

The Antroquinonol B (AnQB) and dehydroeburicoic acid (DeHBA) separately isolated from the Extraction A of Example 1 were added into the culture media of human breast-cancer cells, MCF-7 or MDA-MB-231, to test for tumor cell survival by using anti-cancer drug screen model. This survival assay was carried out with the widely known MTT (3-[4,5-dimethylthiazol-2-yl]2,5-diphenyltetrazolium bromide) assay.

MCF-7 cell line (purchased from B CRC) is originated from primary breast cancer cells (early stage), sensitive to estrogen and therefore is estrogen-dependent, while MDA-MB-231 is insensitive to estrogen and therefore is estrogen-independent, which is a difficult tumor with a low survival rate.

The human breast-cancer cells, MCF-7 and MDA-MB-231 were cultivated in media containing fetal calf serum for 36 hours. The proliferated cells were washed once with PBS, then treated with 1× trypsin-EDTA and centrifuged at 2000 rpm for 25 minutes. The supernatant was discarded and the cell pellet was re-suspended in 30 ml of fresh culture medium by gently shaking. The cells were placed in a 96-well plate.

Subsequently, the plates were read on an ELISA reader to determine the survival rates. The half maximal inhibitory concentration (IC50) values were calculated and results of ex vivo survival assay were thus obtained.

According to those results, it shows that the breast cancer cells are capable of decreasing the survival rate of breast cancer cell MCF-7 and MDA-MB-231 and namely, it is concluded that the Antroquinonol B (AnQB) and dehydroeburicoic acid (DeHBA) can inhibit the growth of breast cancer cell. On the other hand, in a pharmaceutically effective amount of the Antroquinonol B (AnQB) and dehydroeburicoic acid (DeHBA) can be applied to the treatment of breast cancer, or to prevent or to reduce the risk of a breast cancer metastasizing.

### Example 3˜8 Pharmaceutical Compositions of the Invention

The active ingredient of a first agent consisting of dehydroeburicoic acid (DeHBA) and a second agents consisting of Antroquinonol B (AnQB) separated from an Extraction A of Antrodia camphorates of Example 1, was uniformly mixed according to the composition ratio shown in table 1 to be formulated as the pharmaceutical compositions of the present invention used for a prevention and/or treatment of a breast cancer.

TABLE 1
The Pharmaceutical Compositions of The Invention

| | | First Agent | Second Agents |
|---|---|---|---|
| | | (DeHBA) | (AnQB) |
| Example 3 | Composition A (wt. %) | 70 | 30 |
| Example 4 | Composition B (wt. %) | 75 | 25 |
| Example 5 | Composition C (wt. %) | 55 | 45 |
| Example 6 | Composition D (wt. %) | 62 | 38 |
| Example 7 | Composition E (wt. %) | 85 | 15 |
| Example 8 | Composition F (wt. %) | 90 | 10 |

DeHBA: dehydroeburicoic acid
AnQB: Antroquinonol B

By using the same manner as the method described above, respectively human breast cancer cell line MCF-7 and MDA-MB-231 was cultured. After then, the pharmaceutical compositions A˜F of the present invention was applied separately, and then MTT assay for each sample was measured to assess the inhibition effectiveness of various cancer cell activity while using pharmaceutical compositions A˜F of the present invention.

According to those test results of cancer suppression activity, it confirmed that human breast cancer cell line MCF-7 and MDA-MB-231 can be suppressed by the pharmaceutical compositions A˜F of the present invention, particularly all of IC50 (μg/ml) are significantly better than the prior art. In summary, the results show that the pharmaceutical composition of the present invention is a potentially useful drug having pharmaceutical effectiveness in the treatment of various cancers.

What is claimed is:

1. A pharmaceutical composition for treating breast cancer in a subject in need thereof, which comprises
(1) a first agent comprising at least a dehydroeburicoic acid (DeEA) obtained from extractions of a fruiting body or a mycelium of Antrodia camphorata;
(2) a second agent comprising at least an Antroquinonol B (AnQB) obtained from the extractions of a fruiting body or a mycelium of Antrodia camphorata;
wherein the first agent and the second agent shows synergistic effect for use in the treatment of breast cancer or to prevent or to reduce the risk of a breast cancer metastasizing, compared to administration of the first agent or the second agent alone.

2. The pharmaceutical composition as described in claim 1, wherein the first agent is a dehydroeburicoic acid (DeEA) in a pharmaceutically effective amount for use in the treatment of breast cancer, or to prevent or to reduce the risk of a breast cancer metastasizing.

3. The pharmaceutical composition as described in claim 1, wherein the second agent is an Antroquinonol B (AnQB) in a pharmaceutically effective amount for use in the treatment of breast cancer, or to prevent or to reduce the risk of a breast cancer metastasizing.

4. The pharmaceutical composition as described in claim 1, wherein the the first agent and the second agent are in the form of a botanical drug substance (BDS).

5. The pharmaceutical composition as described in claim 1, wherein the dehydroeburicoic acid (DeEA) and/or Antroquinonol B (AnQB) is present in an approximate amount of between 10 mg and 350 mg.

6. The pharmaceutical composition as described in claim 1, wherein the ratio of dehydroeburicoic acid (DeEA) to Antroquinonol B (AnQB) is between about 23:16 to about 16:23 (DeEA:AnQB).

7. The pharmaceutical composition as described in claim 1, wherein the ratio of dehydroeburicoic acid (DeEA) to Antroquinonol B (AnQB) is between about 3:1 to about 1:3 ((DeEA:AnQB).

8. The pharmaceutical composition as described in claim 1, wherein the ratio of dehydroeburicoic acid (DeEA) to Antroquinonol B (AnQB) is between about 9:1 to about 1:9 (DeEA:AnQB).

9. The pharmaceutical composition as described in claim 1, which further comprises a pharmaceutically acceptable ingredient comprising a vehicle, a carrier, a diluent or an excipient.

10. The pharmaceutical composition as described in claim 1, wherein the first agent and the second agent are administered to a human cancer patient.

11. The pharmaceutical composition as described in claim 1, wherein the first agent and the second agent are co-administered.

12. The pharmaceutical composition as described in claim 1, wherein the first agent and the second agent are administered in a daily regimen.

13. The pharmaceutical composition as described in claim 1, wherein the first agent and the second agent compositions are administered in an episodic regimen, intravenous administration, or oral administration.

14. The pharmaceutical composition as described in claim 1, wherein the excipient comprises an ingredient selected from the group consisting of lactose, sucrose, a mannitol, sorbitol, maize starch, wheat starch, rice starch, potato starch, gelatin and tragacanth.

15. The pharmaceutical composition as described in claim 1, wherein further comprises at least one additive selected from the group consisting of absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, bactericides, sweeteners, wetting agents, and a mixture thereof.

16. The pharmaceutical composition as described in claim 1, wherein the breast cancer cells are selected from the group consisting of: breast cancer cell line MCF-7; and breast cancer cell line MDA-MB-231.

17. The pharmaceutical composition as described in claim 1, wherein for use in the treatment of an aggressive breast cancer characterized by overexpression of the Her2 gene.
